(19) United States
(12) Patent Application Publication
(10) Pub. No.: US 2024/0053332 A1
(43) Pub. Date: Feb. 15, 2024

(54) Methods and Compositions Utilising Vitamin B12 Binding

(71) Applicant: Quadram Institute Bioscience, Norwich Norfolk (GB)

(72) Inventors: Simon Carding, Norwich Norfolk (GB); Rokas Juodeikis, Norwich Norfolk (GB)

(21) Appl. No.: 18/039,132

(22) PCT Filed: Nov. 29, 2021
(86) PCT No.: PCT/GB2021/053106

§ 371 Date: May 26, 2023

(30) Foreign Application Priority Data
Nov. 27, 2020 (GB) 2018713.4

(51) Int. Cl. G01N 33/532; G01N 33/82

(52) U.S. Cl. CPC G01N 33/532; G01N 33/82

(57) ABSTRACT

A composition for labelling, detection, immobilisation and/or isolation of at least one target molecule. The composition including a non-covalent affinity, coupling or binding between vitamin B12 binding protein BtuG2 and at least one bond formed between the vitamin B12 and the target molecule. The interaction between BtuG2 and vitamin B12 enables the labelling, immobilisation, isolation and/or detection of the target molecule.

The present invention relates to the attachment of vitamin B₁₂, cobalamin, to proteins, peptides, nucleic acid and other molecules.

The present invention provides compounds, methods, and kits for attaching vitamin B₁₂ to molecules and linking via vitamin B₁₂ binding agents, and refers exclusively to the conjugation of B₁₂ to fluorescent proteins using said binding agent, the person skilled in the art will appreciate that the present invention is not limited to such an fluorescent protein attachment and the affinity can be utilised in a wide range of biotechnological application including labelling and isolation of molecules of interest, as well as diagnostics, probes, affinity matrices and medical devices.

Strong binding partners have the potential to be used as powerful tools in the biosciences, where they can be exploited as probes and affinity matrices. In the case of avidin/biotin this progress stemmed from the ability to chemically modify biotin to allow its attachment to antibodies and other biomolecules.

Most chemical biotinylation reagents consist of a reactive group attached via a linker to the valeric acid side chain of biotin, thereby allowing various conjugation chemistries to yield nonspecific biotinylation of amines, carboxylates, sulphydryls and carbohydrates.

It is therefore an aim of the present invention to provide an improved conjugation chemistries utilising vitamin B₁₂.

In a first aspect of the invention there is provided a composition for labelling, detection, immobilisation and/or isolation of at least one target molecule, said composition including a non-covalent affinity, coupling or binding between vitamin B₁₂ binding protein BtuG2 and at least one bond formed between the vitamin B₁₂ and the target molecule in use, said interaction between BtuG2 and vitamin B₁₂ enabling the labelling, immobilisation, isolation and/or detection of the target molecule.

Preferably the target molecule is a biological molecule. Typically the target biological molecule is a peptide, protein and/or antibody.

In one embodiment the bond between the vitamin B₁₂ and the target molecule is a covalent bond. Typically the target biological molecule is connected via a bond to the primary hydroxyl site of the ribose moiety of vitamin B12.

Many proteins and peptides can be conjugated to vitamin B₁₂. For example, U.S. Pat. No. 5,574,018 teaches vitamin B₁₂ conjugated to erythropoietin, granulocyte colony stimulating factor and consensus interferon through covalent binding at the primary hydroxyl site of the ribose moiety of the vitamin B₁₂.

Conjugates of other bioactive agents and vitamin B₁₂ are taught by Grissom et al. (WO 01/30967 & WO 98/08859). Grissom et al. teach covalent attachment of cancer treatment drugs to the cobalt atom of vitamin B₁₂.

In one embodiment vitamin B₁₂ is not directly linked to the target molecule. Typically the indirect linkage is via a bridging molecule, linking elements or particles.

Typically the target biological molecule is a protein, peptide, nucleic acid, antibody and/or other biological molecule.

In one embodiment vitamin B₁₂ is conjugated to one or more fluorescent protein target molecules. Typically the florescent proteins are GFP, Citrin and/or mCherry.

Typically the conjugated proteins then bind to BtuG2 via vitamin B₁₂. The B₁₂-BtuG2 couple holding large potential for use in a wide range of biotechnological applications.

In one embodiment vitamin B₁₂ is modified or attached to the target molecule by attachment to the 5′ hydroxyl of the ribose of the lower nucleotide loop of cobalamin. Typically it is therefore possible to couple vitamin B₁₂ to a range of proteins or matrices, effectively mimicking the modifications that can be achieved on biotin.

In one embodiment the vitamin B₁₂ is modified or attached to the target molecule by attachment to the secondary alcohol on cobinamide.

In one embodiment a linker compound is used to modify vitamin B₁₂ with a linker compound or element to allow B₁₂ conjugation to a wider range of target molecules and proteins.

In one embodiment the linker compound is succinyl anhydride and the associated chemistry attaching the protein target shown below:

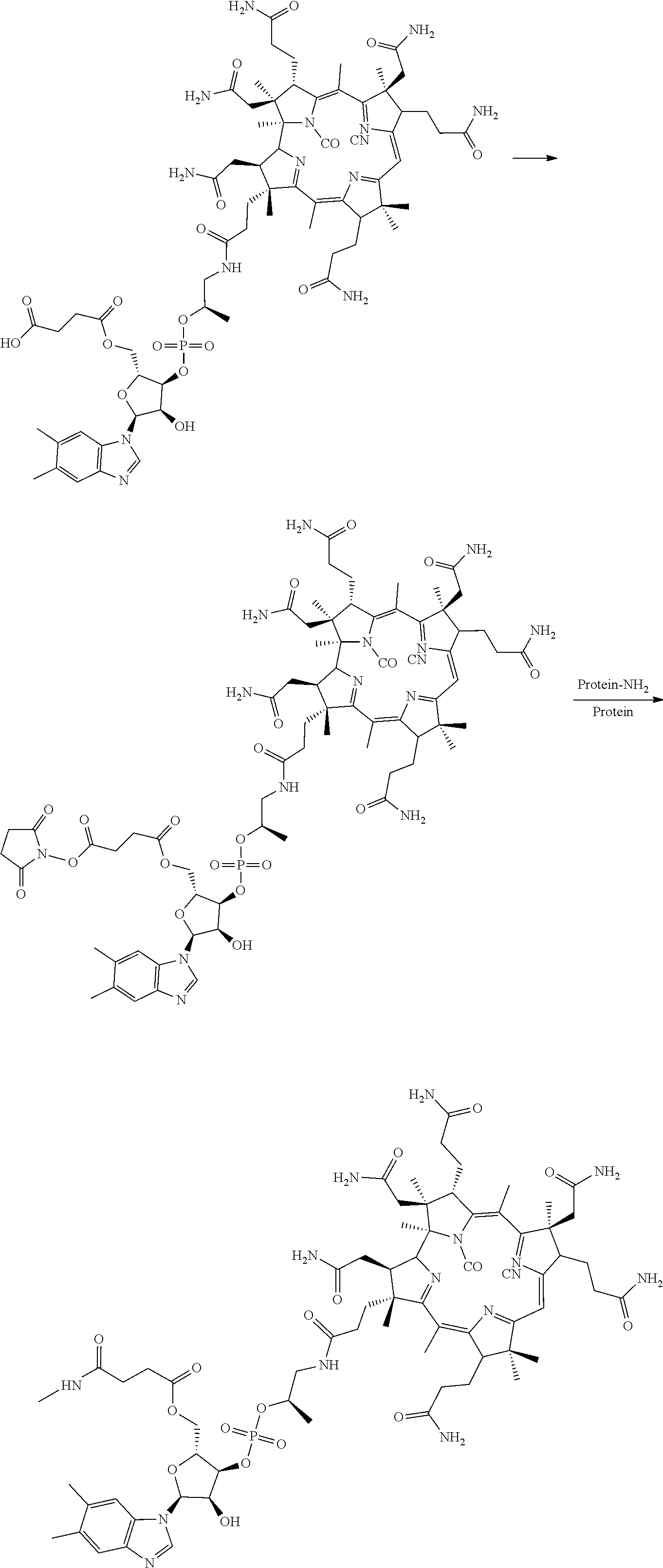

In one embodiment the target biological molecule is covalently attached to a dicarboxylic acid derivative of the primary (5′) hydroxyl group of the ribose moiety of vitamin B₁₂.

In one embodiment the target molecule is bonded to the cobalt of vitamin B₁₂. Typically the bond is a cleavable dative covalent or ionic bond.

In one embodiment a 5′-O-glutaroyl derivative of vitamin B₁₂ is formed by acylation of vitamin B₁₂. Typically the acylation is with a reactive glutaric acid derivative, for example, the anhydride, to selectively convert the primary hydroxyl group (5′-OH) on the .alpha.-ribose moiety to a chemically reactive carboxyl group.

In one embodiment the vitamin B₁₂ acylated derivative is then subsequently reacted with a linker and/or spacer group to form a second derivative. Typically the second derivative in turn is reacted with the target molecule to form a the vitamin B₁₂-target conjugate. Further typically this conjugate is coupled to BtuG2.

In one embodiment the target molecule is attached via a reactive derivative of vitamin B₁₂, said derivative being modified at the primary (5′) hydroxyl group on the ribose moiety to form a chemically reactive carboxyl group. Optionally the target molecule is attached to a further modified vitamin B₁₂, said further modification being the modification of the reactive carboxyl group on the ribose moiety into a mixed acid anhydride, acid halide, or activated ester functional group which is capable of being covalently linked to a target molecule.

In one embodiment a maleimide functionality can be attached to the ribose group allowing the conjugated B₁₂-derivative to interact with free cysteine residues on proteins.

In a preferred embodiment the vitamin B₁₂—target molecule conjugate is recovered, isolated and/or immobilised by association with BtuG2

In a second aspect of the invention there is provided a method of labelling, detecting, immobilisation and/or isolating at least one target molecule, said method including the step of forming at least one bond between vitamin B₁₂ and the target biological molecule and utilising the non-covalent affinity, coupling or binding between vitamin B₁₂ binding protein BtuG2, said interaction between BtuG2 and vitamin B₁₂ enabling the labelling, immobilisation, isolation and/or detection of the target molecule.

A method for preparing a conjugate of vitamin B₁₂ and a target molecule comprising the steps of: a) forming a derivative of vitamin B₁₂ by appending to the primary (5′) hydroxyl group on the ribose moiety a chemically reactive group; b) optionally converting the chemically reactive group on the ribose moiety into a mixed acid anhydride, acid halide, activated ester, or maleimide functional group which is capable of being covalently linked to a target molecule; c) adding the vitamin B₁₂ derivative of step (a) or step (b) with the target molecule to form a conjugate of vitamin B₁₂ and the target molecule; and d) detecting, isolating, immobilising or recovering the conjugate by exposing the same to the vitamin B₁₂ binding protein BtuG2.

Specific embodiment of the invention are now described with reference to the following figures, wherein;

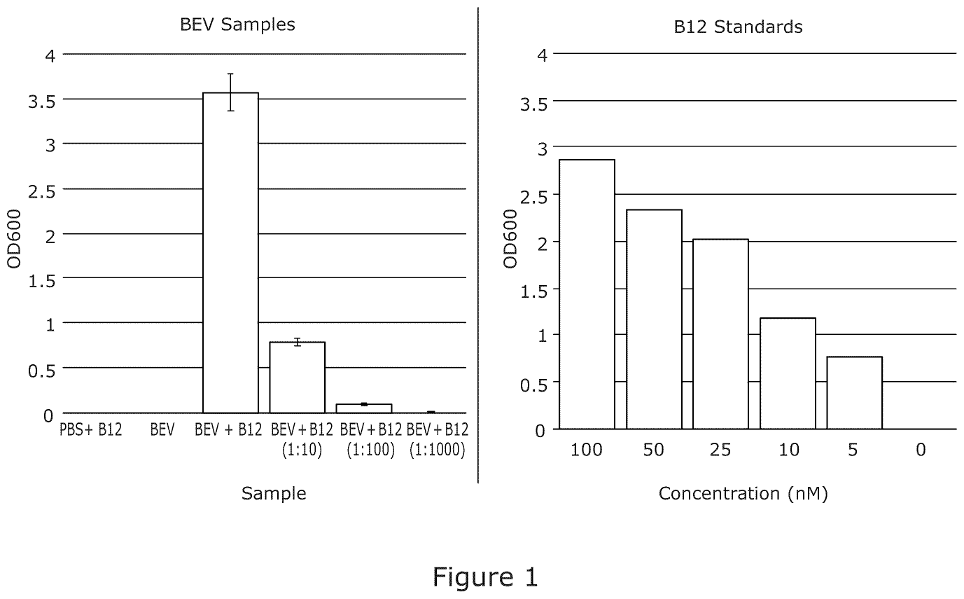

FIG. 1 shows graphs of the results of the growth of Bt being dependent upon vitB₁₂;

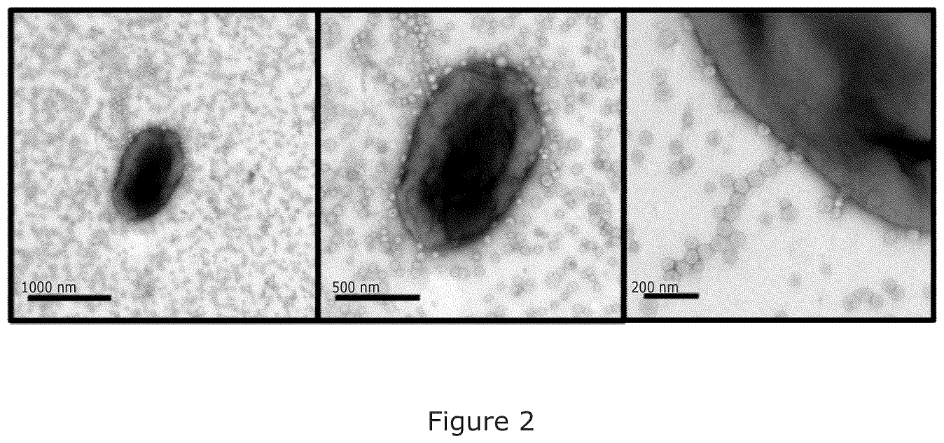

FIG. 2 shows electron micrographs of Bt BEVs; and

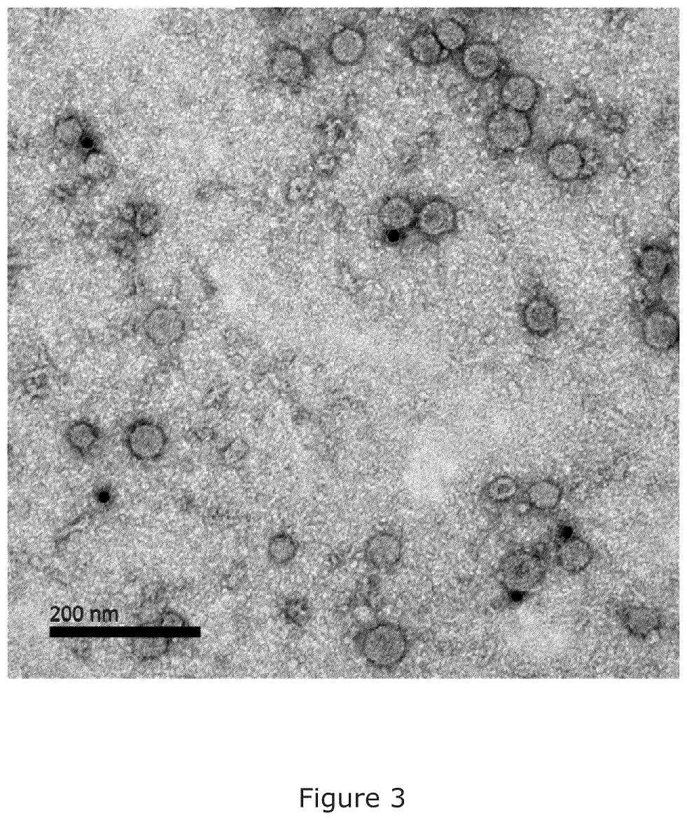

FIG. 3 shows gold label association with the OMVs

In the present invention, the linking of a fluorescent protein to a B₁₂-conjugate has been established, as evidenced by UV-Vis and mass spectral recordings. Conjugated fluorescent proteins with B₁₂ have then been observed to bind to BtuG2 that had been immobilised on a Ni-column.

A maleimide-B₁₂ conjugate has been shown to react with an antibody. The antibody complex has been shown to interact with BtuG2 through an ELISA system. This demonstrates the proof of principle that the BtuG2-B₁₂ binding system can be used in diagnostics.

Furthermore, bacterial extracellular vesicles (BEVs) or outer membrane vesicles (OMVs) generated by Bacteroides thetaiotaomicron (Bt) can provide it with the vitB₁₂ that is essential for its growth.

The right panel of FIG. 1 shows that the growth of Bt is dependent upon vitB₁₂ with no growth possible in its absence (0 nM) with maximal growth at 100 nM vitB₁₂.

The left panel of FIG. 1 shows that BEVs incubated with 1 mM of vitB₁₂ can support the growth of Bt in vitB₁₂-deficient media. Less growth is seen with serial 10-fold dilutions of B₁₂-bound BEVs. By contrast and as controls, no growth is seen by adding 1 mM vitB₁₂ directly to the cultures or by adding BEVs alone with no bound vitB12.

BEVs bind vitB₁₂ in a form that retains its biological activity and can be utilised by parental bacterial cells for sustaining its growth.

We have shown that a B₁₂-conjugated fluorescent protein binds to the Bt OMV via the B₁₂-binding protein BtuG2. Purified OMVs were mixed with the B₁₂-linked mCherry fluorescent protein—and the excess B₁₂ material washed away. The OMVs were then added to an EM grid and cross-reacted with a gold-labelled anti-mCherry antibody. The resulting EM picture in FIG. 3 shows that the gold label is associated with some of the OMVs.

The BtuG2 protein has extremely high affinity for B₁₂, somewhere in the femtomolar region. We believe that it is possible to exploit this tight interaction in the same way as the Biotin-avidin system. We have shown that it is possible to attach protein-linkers to vitamin B₁₂, allow vitamin B₁₂ to become attached to proteins that have the necessary exposed amino acid side chains. Vitamin B₁₂ containing either succinyl anhydride or PFP ester can react with exposed lysine residues, whilst B₁₂ with a maleimide functionality can react with exposed cysteine residues.
(a) We have shown that GFP labelled with B₁₂ will bind to BtuG2. The attachment is specific and strong.
(b) With Mologic, we have shown that a B₁₂-maleimide will bind to a lactate dehydrogenase antibody, allowing the complex to be used in an immunoassay. This provides the proof of principal that the B12-B12 binding protein system can be exploited in therapeutic assays and diagnostics in the same way as biotin-avidin.

1. A composition for labelling, detection, immobilisation and/or isolation of at least one target molecule, said composition including a non-covalent affinity, coupling or binding between vitamin B₁₂ binding protein BtuG2 and at least one bond formed between the vitamin B₁₂ and the target molecule in use, said interaction between BtuG2 and vitamin B₁₂ enabling the labelling, immobilisation, isolation and/or detection of the target molecule.

2. A composition according to claim 1 wherein the target molecule is a biological molecule peptide, protein and/or antibody.

3. (canceled)

4. A composition according to claim 1 wherein the bond between the vitamin B₁₂ and the target molecule is a covalent bond.

5. A composition according to claim 1 wherein the target biological molecule is connected via a bond to the primary hydroxyl site of the ribose moiety of vitamin B₁₂.

6. A composition according to claim 1 wherein vitamin B₁₂ is not directly linked to the target molecule.

7. A composition according to claim 6 wherein the indirect linkage is via a bridging molecule, linking elements or particles.

8. A composition according to claim 1 wherein vitamin B₁₂ is conjugated to one or more fluorescent protein target molecules.

9. A composition according to claim 1 wherein the conjugated proteins then bind to BtuG2 via vitamin B₁₂.

10. A composition according to claim 1 wherein vitamin B₁₂ is modified or attached to the target molecule by attachment to the 5′ hydroxyl of the ribose of the lower nucleotide loop of cobalamin.

11. A composition according to claim 1 wherein the vitamin B₁₂ is modified or attached to the target molecule by attachment to the secondary alcohol on cobinamide.

12. A composition according to claim 1 wherein a linker compound is used to modify vitamin B₁₂ with a linker compound or element.

13. (canceled)

14. A composition according to claim 1 wherein the target biological molecule is covalently attached to a dicarboxylic acid derivative of the primary (5′) hydroxyl group of the ribose moiety of vitamin B₁₂.

15. A composition according to claim 1 wherein the target molecule is bonded to the cobalt of vitamin B₁₂.

16. (canceled)

17. A composition according to claim 1 wherein a 5′-O-glutaroyl derivative of vitamin B₁₂ is formed by acylation of vitamin B₁₂.

18. (canceled)

19. (canceled)

20. (canceled)

21. (canceled)

22. A composition according to claim 1 wherein the target molecule is attached via a reactive derivative of vitamin B₁₂, said derivative being modified at the primary (5′) hydroxyl group on the ribose moiety to form a chemically reactive carboxyl group.

23. A composition according to claim 22 wherein the target molecule is attached to a further modified vitamin B₁₂, said further modification being the modification of the reactive carboxyl group on the ribose moiety into a mixed acid anhydride, acid halide, or activated ester functional group which is capable of being covalently linked to a target molecule.

24. A composition according to claim 23 wherein a maleimide functionality can be attached to the ribose group allowing the conjugated B₁₂-derivative to interact with free cysteine residues on proteins.

25. A composition according to claim 1 wherein the vitamin B₁₂—target molecule conjugate is recovered, isolated and/or immobilised by association with BtuG2

26. A method of labelling, detecting, immobilisation and/or isolating at least one target molecule, said method including the step of forming at least one bond between vitamin B₁₂ and the target biological molecule and utilising the non-covalent affinity, coupling or binding between vitamin B₁₂ binding protein BtuG2, said interaction between BtuG2 and vitamin B₁₂ enabling the labelling, immobilisation, isolation and/or detection of the target molecule.
